(19) United States
(12) Patent Application Publication
(10) Pub. No.: US 2006/0166295 A1
(43) Pub. Date: Jul. 27, 2006

(54) Method for determination of likelihood of occurrence of preterm labor in pregnant females

(76) Inventors: James Woods, Rochester, NY (US); Marion W. Andres, Rochester, NY (US); Lin Xu, Rochester, NY (US)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 10/519,884

(22) PCT Filed: Jun. 30, 2003
(86) PCT No.: PCT/US03/20699

§ 371 Date: Jul. 15, 2005

Related U.S. Application Data
(60) Provisional application No. 60/393,182, filed on Jul. 1, 2002.

(51) Int. Cl. G01N 33/537

(52) U.S. Cl. 435/7.92

(57) ABSTRACT

The presence of 3-chlorotyrosine in vaginal secretions in a predictor of the likelihood preterm premature rupture of the fetal membranes, i.e., the chorioamnion and/or the occurrence of the risk of preterm labor. 3-chlorotyrosine is a marker for the excessive production of hypochlorous acid which causes focal areas of increased collagen destruction in the chorioamnion, preterm premature rupture of membranes, or increased production of postaglandin and preterm labor. The inventive method comprises determining the likelihood of the occurrence of preterm premature rupture of membranes or increased production of postaglandin and preterm labor in a pregnant female by obtaining a sample of the females vaginal secretions, and analyzing the secretions for the presence and amount of 3-chlorotyrosine in the sample. Various antibody-based tests can be used to measure 3-chlorotyrosine, and a number of neoantigens, useful in raising antibodies to 3-chlorotyrosine, are disclosed.

### BACKGROUND OF THE INVENTION

1. Area of the Art

This invention relates to the field of accurate identification of pregnant women who are at risk for the occurrence of preterm labor giving birth to extremely low weight babies with low likelihood chance of survival.

2. Description of the Prior Art

Preterm labor (PTL) leading to premature birth is a leading cause of both infant mortality and long-term disabilities. While there have been some treatments proposed (see U.S. published Patent Application 2001/0031731 to Buhimschi et al., the contents of which are incorporated herein by reference), there is a serious problem in deciding when such treatments should be instituted and whether the treatment is at all effective. Especially as it is not clear that all of the possible PTL treatments are without potential harm. Thus, one must be reluctant to institute a treatment where the need for treatment is uncertain. One predictor for PTL is previous instances of PTL in the same individual. However, that is somewhat like closing the barn door after the escape. It would be much better (and cost effective) to detect actual markers of PTL.

Currently, fetal fibronectin (fFN) in vaginal secretions is the principal FDA approved biomarker for PTL. Fetal fibronectin is an adhesion molecule that binds the amnion layer in the fetal membrane to the chorion layer beneath it. Typically, fFN can be detected in vaginal secretions early in pregnancy (<20 weeks' gestation), reflecting the fact that the chorion and amnion do not fuse until 20 weeks. From 20 to 34 weeks' gestation, no fFN is detected in vaginal secretions. After 34 weeks' gestation, fFN begins to appear, presumably reflecting some separation of chorion and amnion as the collagen matrix remodels in preparation for labor at term. During this time, MMP-9, the enzyme that degrades collagen IV in basement membranes, increases ultimately to weaken the fetal membranes for the birth process.

Fetal fibronectin, when detected in <34 weeks' gestations, has been interpreted to indicate an increased risk of PTL. Presumably the agitation that occurs as premature labor begins produces shearing forces that lead to release of fFN into the vaginal secretions. Depending on the study, the presence of fFN in vaginal fluid is associated with a 20 to 50% chance of PTL. On the other hand, absence of fFN is associated with a 99% chance of not going into labor prematurely. This test, then, is much more useful for its negative predictive value than its positive predictive value.

Analysis of the chorioamnion shows that its strength is the result of collagen. Collagen is produced by fibroblasts in the amnion and chorion. Results of culturing amnion epithelial cells indicate that there is active biosynthesis and secretion of collagenous matrix up to term (1). Five types of collagen have been identified, types I, III, IV, V and VI (out of 12 known collagen types). Types I, III, V and VI are organized in triple helices. The strength for these collagens is derived from their helix configurations and hydroxyproline and hydroxylysine bridges across the helix. Type IV collagen is different from the other four types in that it forms a mesh as part of the basement membrane (21).

Matrix metalloproteinase I (MMP-1) degrades types I, II, and III collagens; MMP-2 and MMP-9 degrade type IV collagen (31). MMP-3, 7, 10, and 11 have broad substrate specificity. Release of matrix metalloproteinase is controlled by tissue inhibitors of metalloproteinases or TIMPS that bind with metalloproteinases and prevent them from degrading the collagen (21).

Tensile strength and thickness measurements of the chorioamnion indicate that the membranes are thinner at the rupture site than over the placenta (Artel 1976 supra). Moreover, preterm membranes in general are stronger than term PROM or spontaneous rupture of membrane (SROM) membranes suggesting that PPROM represents a local defect (15).

Metalloproteinase 9 (which degrades collagen IV) has been shown to be upregulated naturally at term in association with a reduction in membrane tensile strength (27).

Infection, or culturing membranes with Escherichia coli lipopolysaccharide or group A streptococcus polysaccharide also has been shown to increase MMP 9 (7). Increased MMP-9 also was noted when chorioamnion segments were cultured with the ROS, superoxide (5).

It would appear that Inflammation, therefore, plays a role in PPROM. Increased MMP-9 is found in amniotic fluid of term PROM patients and those in labor but not in patients undergoing elective cesarean section (28). Likewise, MMP-3 in amniotic fluid is increased three-fold in PPROM patients over term controls (8) Stromelysins in placental membranes and amniotic fluid with premature rupture of membranes. Others have reported that MMP-1 is increased in amniotic fluid of PPROM patients and is even higher if bacteria are present in the fluid (17).

### SUMMARY OF THE INVENTION

We have discovered an improved method for predicting the likelihood of the occurrence of preterm premature rupture of the fetal membranes (PPROM), i.e., the chorioamnion and/or the occurrence of the risk of preterm labor (PTL). The inventive method provides greater accuracy than the currently available procedure using the detection of fFN.

More particularly, we have discovered that the excessive production of the reactive oxygen species (ROS), hypochlorous acid (HOCl), or excessive decreases of antioxidant defenses lead to focal areas of increased collagen destruction in the chorioamnion and preterm premature rupture of membranes (PPROM), or increased production of prostaglandin and preterm labor (PTL). Increased dietary intake of vitamins C and E together offers defense against HOCl-induced membrane damage and prostaglandin production and may prevent PPROM and PTL (32, 33).

The inventive method comprises determining the likelihood of the occurrence of preterm premature rupture of membranes or increased production of prostaglandin and preterm labor in a pregnant female by obtaining a sample of the females vaginal secretions, and analyzing the secretions for the presence and amount of hypochlorous acid by measuring the amount of 3-chlorotyrosine in the sample.

### BRIEF DESCRIPTION OF THE FIGURES

FIG. 1 shows the steps of producing an N-acetyl-3-chlorotyrosine containing antigen for subsequent antibody production.

FIG. 2 shows the steps of producing an N-acetyl-3-chlorotyrosine and an N-acetyl-3,5-dichlorotyrosine containing antigen for subsequent antibody production.

FIG. 3A shows the first three steps of producing a novel 3-chlorotyrosine containing hapten (3-(3-chloro-4-hydroxy-benzyl)-6-mercaptomethyl-piperazine-2,5-dione) which is ideal for coupling to a carrier protein to form an antigen for subsequent antibody production.

FIG. 3B shows the next three steps of producing a novel 3-chlorotyrosine containing hapten (3-(3-chloro-4-hydroxy-benzyl)-6-mercaptomethyl-piperazine-2,5-dione) which is ideal for coupling to a carrier protein to form an antigen for subsequent antibody production.

FIG. 3C shows the final steps of producing a novel 3-chlorotyrosine containing hapten (3-(3-chloro-4-hydroxy-benzyl)-6-mercaptomethyl-piperazine-2,5-dione) which is ideal for coupling to a carrier protein to form an antigen for subsequent antibody production.

### DETAILED DESCRIPTION OF THE INVENTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide a method using 3-chlorotyrosine in vaginal secretions to diagnose an increased likelihood of preterm labor.

Reactive oxygen species are tissue damaging molecules used by phagocytes to kill bacteria and which leak from the electron transport system of the mitochondria during cell respiration. They are characterized either by a single unpaired electron in the outer orbit (examples: superoxide, hydroxyl radical, and nitric oxide) or as molecules that share an electron in their outer orbit (examples: hypochlorous acid, hydrogen peroxide). Their tissue damaging actions result in part as they aggressively seek to extract an electron from an adjacent molecule to recreate electron stability. Hydrogen atoms in the double bonds or tail of polyunsaturated fats offer available targets for this form of extraction, thereby setting the stage for lipid peroxidation as covalent bonds are disrupted. ROS also denature proteins, damage DNA, adversely alter collagen and disrupt the integrity of cell membranes (10).

There are certain clinical states that are associated with PPROM known to produce ROS or consume antioxidants. These include:
1. Infection: Chronic infection or inflammation most likely is part of each PPROM case. In-vitro studies indicate that metalloproteinase-producing bacteria such as Pseudomonas aeruginosa, Staphylococcus aureus, and Bacteroides melaninogenicus can decrease bursting load and work-to-rupture in the fetal membrane. Studies also have shown that Staphylococcus aureus and group B streptococci can decrease chorioamnion tensile strength. In one study, when membranes were exposed to activated neutrophils, similar findings were documented which were augmented further in the presence of Staphylococcus aureus (18, 25, 19).
2. Cigarette smoking: Smoking is a leading risk factor for PPROM. Tobacco smoke contains a complex mixture of ROS that induce systemic oxidative damage to multiple tissues. At the level of the chorioamnion, cigarette smoking most likely consumes antioxidants, thereby making the tissues more vulnerable to the normal process of ROS generation (24).
3. Second trimester bleeding: Second trimester bleeding probably increases the risks of PPROM by providing a fertile medium for bacterial growth or by releasing iron as red cells degrade. Iron, in turn, could catalyze formation of ROS.
4. Cocaine use: The relationship of cocaine use to PPROM is believed to result from ischemia and then reperfusion caused by the drug and which generates free radicals as reperfusion follows the ischemic process (37).

ROS can degrade collagen in vitro. Hypochlorous acid, the principal ROS released by phagocytes, is considered the main ROS operative in the generation of PPROM and PTL. Hypochlorous acid damages collagen in the chorioamnion by blocking the tissue inhibitors of the metalloproteinases, thereby upregulating their activity to result in collagen degradation. Hypochlorous acid also attacks the proline and 4-hydroxyproline sites that normally provide cross linkages in the collagen helix as support systems (20).

Membrane segments, when incubated with varying doses of hypochlorous acid, demonstrate decreased collagen 1 staining and alterations in the amnion epithelium. Similar findings have been observed when chorioamnion segments have been incubated with superoxide but since this is a much weaker ROS, the tissue damage is less than that observed with hypochlorous acid (22, 12).

In other tissues, hypochlorous acid alters a range of otherwise normal cellular functions. When red blood cell membranes are exposed to hypochlorous acid, cell membrane fluidity and membrane Na⁺/K⁺/Mg⁺⁺ ATPase activity are compromised (36).

Epithelial amnion cells also react in other ways to exposure to reactive oxygen species. In vitro studies demonstrate that monolayers of amniocytes, when exposed to the ROS exhibit increased intracellular calcium, decreased intracellular magnesium, and release of arachidonic acid (precursor for prostaglandin production in the inflammatory pathway). When the increase in intracellular calcium is prevented, release of arachidonic acid is decreased (16). Amniocytes exposed to tumor necrosis factor alpha and interleukin-1 also demonstrated increased production of MMP, and prostaglandin E2 (26). These findings suggest a possible relationship between PPROM and preterm labor.

The body has a number of antioxidant defenses, which are capable of scavenging reactive oxygen species, and to minimize or prevent ROS-tissue induced damage. Enzyme antioxidants also scavange ROS. Superoxide dismutase (SOD) converts superoxide to hydrogen peroxide. Catalase or glutathione peroxidase converts superoxide to hydrogen peroxide or oxygen gas (O₂) and water, respectively. Other important antioxidants like albumin, uric acid, and bilirubin bind trace metals thereby preventing them from participating in free radical production.

Dietary antioxidants offer a different level of protection. Ascorbic acid (vitamin C), a water soluble vitamin, cannot be synthesized in the human body and is obtained by consuming fruits and vegetables such as red and yellow peppers, broccoli, strawberries and oranges. Vitamin E (tocopherol-OH) is a lipid soluble antioxidant and the most important chain-breaking defense against lipid peroxidation. It is found in plant oils and nuts. Vitamin C and vitamin E now are believed to work synergistically. Vitamin E, by donating a hydrogen atom, blocks the progression of lipid peroxidation but becomes a free radical (tocopherol-O.). Ascorbic acid then donates a hydrogen atom to the tocopheryl radical, thereby recycling it back into the lipid interface as tocopherol-OH; in this process ascorbic acid becomes dehydroascorbic acid, a weak radical which is excreted in the urine. As long as adequate vitamin C is available, vitamin E is continually recycled (11).

In vitro evidence has shown that vitamin C and E can prevent ROS-induced damage to the chorioamnion. In one series, membrane segments were incubated for four hours in graded dose of hypochlorous acid. This level of exposure produced damage to collagen 1 and alterations in amnion epithelium architecture. When these membranes were incubated first with vitamins C and E, rinsed, and then exposed to hypochlorous acid, no ROS-induced damage was detected. This is the first evidence that vitamin C and vitamin E may offer protection to the chorioamnion from ROS-induced exposure (22).

Recent data indicate that vitamins C and E distribute through the maternal-fetal-amniotic fluid compartments differently. For these studies, plasma vitamin E concentrations were determined by reversed phase HPLC and standardized to cholesterol. Vitamin C was determined by the 2,4-DNPH method. The results indicate that vitamin E is higher in the maternal plasma than fetal plasma. For vitamin C, the distribution is different. Amniotic fluid contains the highest concentrations of vitamin C with less being noted in fetal plasma and the lowest concentrations in maternal plasma (34).

Low maternal plasma and leukocyte ascorbic acid concentrations (vitamin C) have been linked to PPROM. Specifically, plasma ascorbic acid levels at six to eight months of pregnancy were lower in patients at term experiencing PROM as compared with patients entering labor with intact membranes (29).

Low leukocyte vitamin C levels at 20 weeks' gestation also were associated with an increase in PPROM (6). In amniotic fluid, significantly lower levels of ascorbic acid have been found in smokers than nonsmokers (3). Studies of vitamin E concentrations and their relationship to PPROM have not been published.

Many investigators believe that women in the United States are not taking adequate levels of vitamins C and E in their prenatal vitamins. Prenatal vitamins contain 60 to 100 mg of vitamin C and 10 to 30 IU of vitamin E. The dosage of vitamin C was established initially to prevent scurvy; the dosage of vitamin E was chosen which matched that taken in with a normal diet. Subsets of the population are poorly supplemented with dietary vitamin C and E. Data from the first NHANES (1971-1974) showed that African Americans had four-fold lower plasma concentrations for vitamin E and ten-fold lower plasma concentrations for vitamin C than Caucasians (Block 1988). If vitamins are to be used as therapy and not simply to combat dietary deficiencies, then dosages of vitamins C and E five to ten times those found in prenatal vitamins must be studied.

Hypochlorous acid produced by neutrophils, is an initiating event in the cascade leading to release of arachidonic acid from amnion epithelial cells and the subsequent production of prostaglandins leading to PTL. Hypochlorous acid (HOCl) is a primary biomarker for neutrophil-derived inflammation. HOCl is formed as myeloperoxidase, a major phagocytic protein, catalyzes the 2-electron peroxidation of chloride. In fact, myeloperoxidase is the only human enzyme capable of producing HOCl at physiologic concentrations of halide ions. Since halogenated molecules are formed by such limited pathways, they serve as good markers for phagocytic-predicted tissue damage (14, 30).

We have discovered that the measurement of hypochlorous acid in vaginal secretions of women at risk for preterm labor, provides a valuable biomarker. Unfortunately, hypochlorous acid is not sufficiently stable to be detectable at a distant site. 3-chlorotyrosine, however, is a stable oxidized product generated from hypochlorous acid and in accordance with the present invention, it can be detected and measured in the vaginal secretions of women at risk for PTL and provide an accurate determination of the presence and amount of HOCl in the secretions. This, in turn, provides an accurate indicator of the likelihood of PTL and/or PPROM so that therapeutic measures can be taken.

Because of its unique pathway for formation, 3-chlorotyrosine detected in vaginal secretions reflects the release of HOCl further up in the pregnant uterus. 3-chlorotyrosine has been detected in elevated levels in patients with coronary artery disease when low density lipoprotein (LDL) is exposed to HOCl but not when LDL is exposed to other ROS such as OH., copper, iron, heme, glucose, peroxynitrite, horseradish peroxidase, lactoperoxidase or lipoxyglucose. It appears that 3-chlorotyrosine is a stable marker of LDL oxidation by HOCl. Moreover, it is increased markedly in atherosclerotic tissue obtained at vascular surgery and is mildly increased in plasma LDL of older men with coronary artery disease but not healthy young men (13).

The presence and level of the 3-chlorotyrosine in the sample of vaginal secretion can readily be determined by immunoassay utilizing either polyclonal or monoclonal antibodies to the 3-chlorotyrosine modified proteins. Radioimmunoassays and enzyme immunoassays, e.g., an enzyme-linked immunosorbent assay (ELISA)) known in the art, see for example (9).

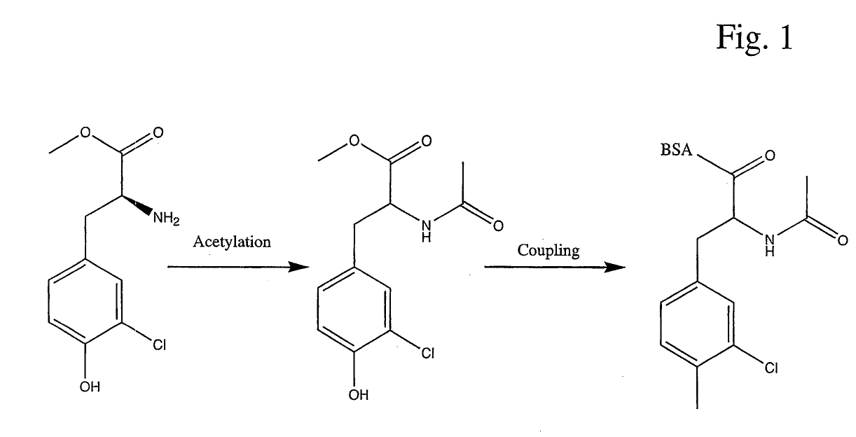

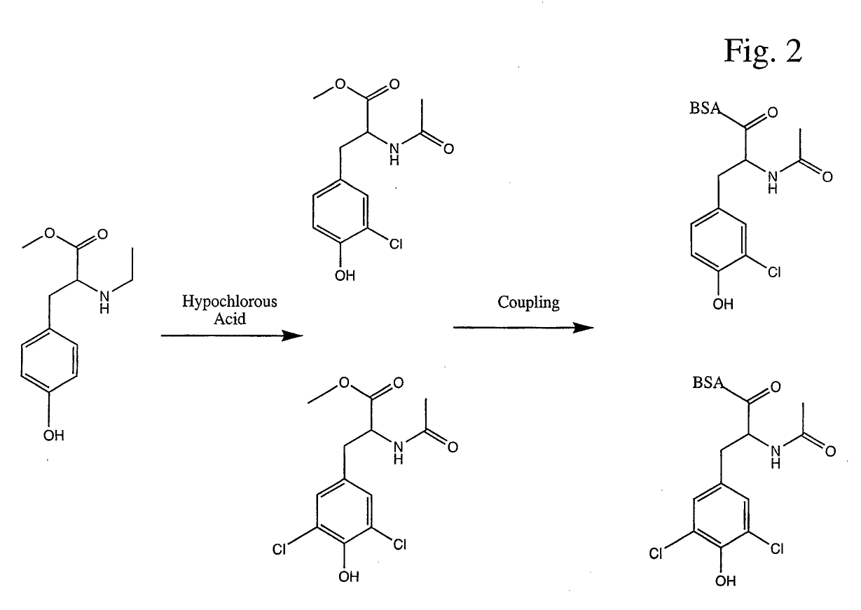

In carrying out the procedure, a capture ELISA or a sandwich ELISA is used for the detection and quantitative analysis of 3-chlorotyrosine in a clinical sample. This involves as a first step, raising antibodies against 3-chlorotyrosine coupled to a carrier such as bovine serum albumin (BSA) because 3-chlorotyrosine itself is a hapten. As shown in FIG. 1, most standard procedures for coupling the hapten to protein require first protecting the amino group of 3-chlorotyrosine—for example by acetylation. This may be carried out by chemical means, e.g., Schotten-Baumann reaction. Those of ordinary skill in the art will recognize that a variety of other amino protective reagents can be employed. N-acetyl-3-chlorotyrosine (N-acetyl-3-CIY) is then coupled to the BSA using any of a number of well-know linking reagents. The drawing shows coupling though the carboxyl group of the modified tyrosine, which coupling can be carried out, for example, by use of carbodiimide reagents, as is well known to those of ordinary skill in the art. FIG. 2 shows an alternative approach in which treatment of N-acetyl-tyrosine with hypochlorous acid produces both N-acetyl-3-chlorotyrosine and N-acetyl-3,5-dichlorotyrosine both of which are subsequently coupled to a carrier protein. Both the monochloro and dichloro product are found in vaginal secretions although the former is much more abundant. By using both compounds to form antigens, the resulting antibodies show enhanced sensitivity while remaining highly selective.

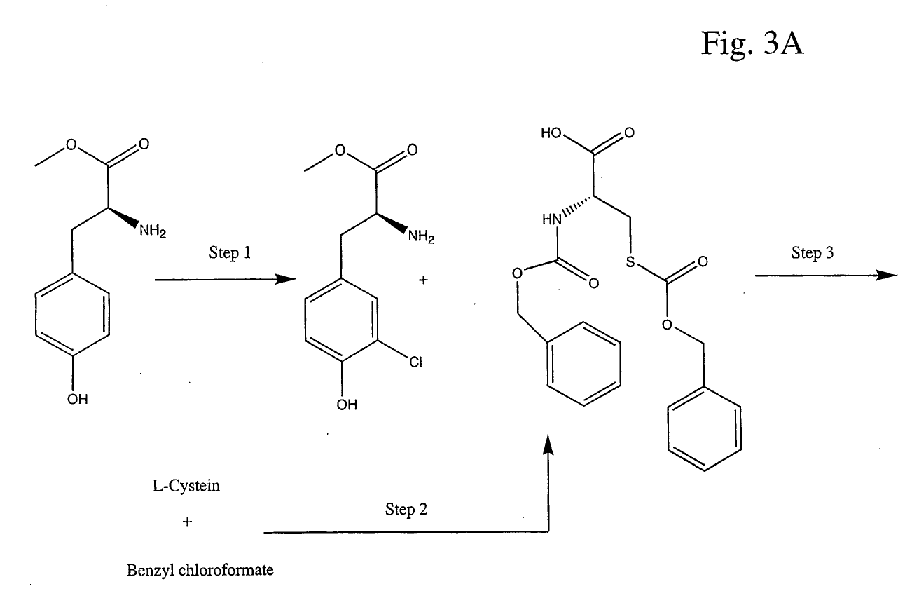

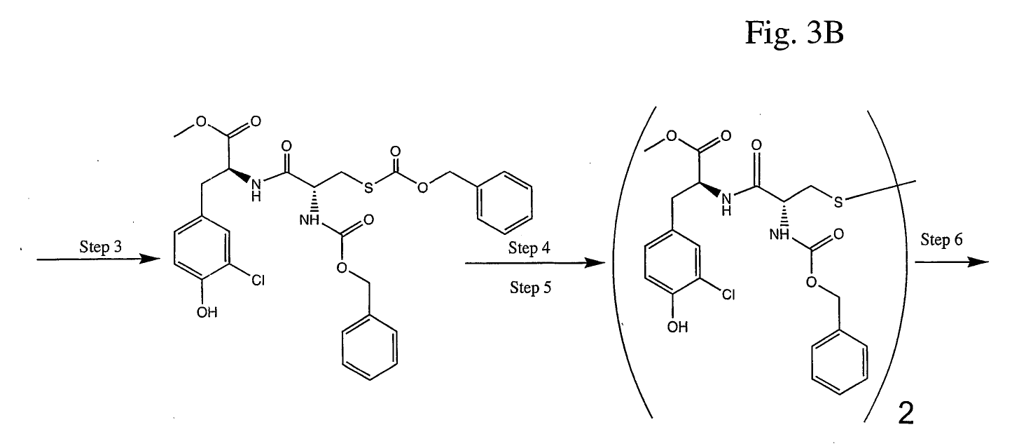

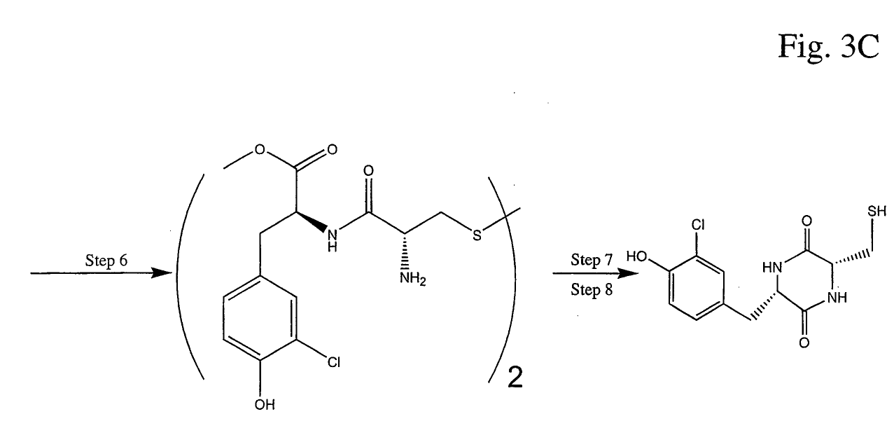

FIG. 3 (FIGS. 3A, 3B, and 3C) shows the synthesis of a novel hapten (3-(3-chloro-4-hydroxy-benzyl)-6-mercaptomethyl-piperazine-2,5-dione) which is optimized for sulfhydryl linking to a carrier protein to produce an effective neoantigen for raising antibodies to 3-chlorotyrosine. The illustrated synthesis combined with linkage to a protein modified with such well-known N-hydroxysuccinimide-maleimide containing linkers as SMCC (4-(Maleimidomethyl)cyclohexanecarboxylic Acid N-Hydroxysuccinimide Ester) allows simple creation of an antigen having 3-chlorotyrosine epitopes. Note that in the figures the carrier protein is indicated at BSA (bovine serum albumin) but other suitable carrier proteins such as thyroglobulin and keyhole limpet hemocyanin can be used instead.

According to FIG. 3A in a first synthetic step chlorine gas is bubbled into an ice-cold methanol solution of tyrosine methyl ester (see, Y. Terada, Tetrahedron Lett. 1996, 37(48):8791-8794).

In a second step benzyl choloroformate and sodium hydroxide solution were added simultaneously to a solution of L-cysteine in sodium hydroxide to yield N,S-dicarbobenzoxy-L-cysteine at 90% yield (see, A. Berger, J. Niguchi, E. Katchalski, J. Am. Chem. Soc. 1956, 78:4483-4487).

In a third step the addition product, N,S-dicarbobenzoxy-L-cysteine, and the 3-chlorotyrosine methyl ester are reacted for twelve hours with dicyclohexylcarbodiimide in N,N-dimethyl-formamide (see, K. Jost, J. Rudinger, F. Sorm, Coll. Czech. Chem. Commun. 1961, 26:2496-2510) to give a 90% yield of a dipeptide N,S-dicarbobenzoxy-L-cysteinyl-L-3-chlorotyrosine methyl ester.

The dipeptide (see FIG. 3B) is then treated in a fourth step with sodium methoxide in methanol (see, L. Zervas, I. Photaki, N. Ghelis, J. Am. Chem. Soc. 1963, 85:1337-1341) for 10 min. and in a fifth step the reaction mixture was acidified with acetic acid and titrated with iodine in methanol to yield the dimer N,N′-biscarbobenzoxy-L-cystinyldi(3-chloro)tyrosine dimethyl ester at 90% yield (see, L. Zervas, I. Photaki, N. Ghelis, J. Am. Chem. Soc. 1963, 85:1337-1341).

The dimer is then treated in a sixth step for 15 min. with 30% hydrobromic acid in acetic acid solution (see, K. Blaha. Coll. Czech. Chem Commum. 1969, 34:4000-4005) to give an 89% yield of L-cystinyldi(3-chloro)tyrosine dimethyl ester.

The L-cystinyldi(3-chloro)tyrosine dimethyl ester is then treated in a seventh step for eight hours with ammonia in methanol (see, E. Fischer, Chem Ber. 1906, 39:2893) to give a 60% yield. This product (see FIG. 3C) is then treated in an eighth step with tri-n-butylphosphine in aqueous methanol to cleave the cystine-disulfide bond (see, S. A. Khan, B. W. Erickson, J. Am. Chem Soc. 1981, 103:7373-7376) to give a 78% yield of the final product, 3-(3-chloro-4-hydroxy-benzyl)-6-mercaptomethyl-piperazine-2,5-dione (overall yield of about 30%).

These antigens are advantageous because a single (or dual) neoantigen is used which allows a high degree of selectivity for the detection of protein-bound 3-chlorotyrosine. In the prior art some use has been made of hypochlorous acid exposed proteins as antigens. This provides less selective antibodies because such proteins are likely to contain other modified amino acids including modified lysine, cysteine, and histidine residues are obtained.

Using the antigens described above, the antibodies are raised for example in a laboratory animal such as rabbit. This can be carried out by injecting the antigen followed by a booster inoculation with the same protein (usually, at least four weeks after priming) and collecting antisera from the animal after an additional period of four weeks. Alternatively, these antigens can be used to raise monoclonal antibodies using methods well known to those of ordinary skill in the art. Subsequently, the antisera may optionally fractionated using chromatographic and similar techniques to yield purified IgG.

A fraction of the antibody obtained. (e.g., the rabbit antisera diluted to 2 μg per microtiter well in phosphate buffered saline (PBS)) is then bound to standard ELISA plates by overnight incubation at 4° C. The plates are washed with PBS containing a surfactant such as 0.05% Tween 20 (polysorbate 20). The clinical sample to be analyzed is appropriately diluted, and preferably serially diluted samples, (10-100 μl/well) are added to ELISA plates (about 100 μl/well)

After 1 hour incubation at 37 ° C., the plates are washed 3-4 times. Then a suitable amount of a secondary antibody (depending on the source of the primary antibody—e.g., if the primary antibody is raised in rabbits, then goat-antirabbit antibody is used as the secondary antibody) conjugated to a fluorescent or chemiluminescent label or an enzyme such as alkaline phosphatase or peroxidase is added. The secondary antibody can be monoclonal or a polyclonal antibody.

After washing the plate with PBS, about 50 μl of an appropriate reaction or substrate solution (in the case of a chemiluminescent or an enzyme label) is added per well. After a 1 hour reaction period, the absorbance at a suitable wavelength (e.g., 450-590 nm) is measured using a plate reader or by optical density scanning of the plate. Fluorescent labels can be read directly by a fluorescence plate reader.

The ability to measure 3-chlorotyrosine, a stable biomarker of hypochlorous acid in vaginal secretions of at-risk women for preterm labor, significantly increases our ability to detect these at-risk women and implement labor-inhibiting medications including antioxidant compounds intended to block the action of hypochlorous acid. Treatment can be instituted only when actually needed. Further, by monitoring the 3-chlorotyrosine it is possible to see if the treatments are having a positive effect. Various other immunoassays and detection techniques will be apparent to those skilled in the art after reading the present disclosure without departing from the spirit and scope of the invention. For example, it is known to utilize mass spectrometry to measure 3-chlorotyrosine in human tissue with attomole sensitivity (14). It is intended that all such other techniques for measuring 3-chlorotyrosine in vaginal secretions be included within the scope of the present invention.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

### LITERATURE CITED

1. Aplin J D, Campbell S, Donnai P, Bard J B L, Allen T D. Importance of vitamin C in maintenance of the normal amnion: An experimental study. Placenta 1986; 7:377-389.
2. Artal R, Sokol R J, Neuman M, Burstein A H & Stojkov J. The mechanical properties of prematurely and non-prematurely ruptured membranes. Am J. Obstet Gynecol 1976; 125:655-659.
3. Barrett B, Gunter E, Jenkins J, Wang M: Ascorbic acid concentration in amniotic fluid in late pregnancy. Biol Neonate 1991; 60:333-335.
4. Block G, Cox C, Nadans J, Schreiber G B, Licitra L, Melia N. Vitamin supplement use by demographic characteristics. Am J Epidemiol. 1988; 127:297-309.
5. Buhimschi I A, Kramer W B, Buhimschi C S, Thompson L P and Weiner C P (2000) Reduction-oxidation (redox) state regulation of matrix metalloproteinase activity in human fetal membranes. Am J Obstet Gynecol, 182, 458-464.
6. Casanueva E, Avila-Rosas H, Polo E, Tejer E, Narcio-Reyes M C, Pfeffer F. Vitamin C status, cervico-vaginal infection and premature rupture of amniotic membranes. Arch Med Res 1995; 26:5149-52.
7. Fortunato S J, Menon R, Lombardi S J. Collagenolytic enzymes (gelatinases) and their inhibitors in human amniochorionic membrane. Am J Obstet Gynecol 1997; 177:731-741.
8. Fortunato S J, Menon R, and Lombardi S J (1999) Stromelysins in placental membranes and amniotic fluid with premature rupture of membranes. Obstet Gynecol, 94, 435-440.
9. Klonisch T, Panayotou G, Edwards P, Jackson A M, Berger P, Delves P J, Lund T, Roitt I M. Enhancement in antigen binding by a combination of synergy and antibody capture. Immunology. 1996; 89 (2):165-71.
10. Halliwell B, Gutteridge J M C, Cross C E. Free radicals, antioxidants, and human disease: Where are we now? J Lab Clin Med 1992; 19:598-620.
11. Hamilton I M, Gilmore W A S, Benzie I F, Mulholland C W & Strain J J. Interactions between vitamins C and E in human subjects. Br J Nutr. 2000; 84:261-267.
12. Hammer A, Desoye G, Dohr G, Sattler W, and Malle E, Myeloperoxidase-Dependant generation of Hypochlorite-modified Proteins in Human Placental Tissues during Normal Pregnancy, Laboratory Investigation, 81, 1543-554
13. Hazen S L, Heinecke J W. 3-chlorotyrosine, a specific marker of myeloperoxidase-catalyzed oxidation, is markedly elevated in low density lipoprotein isolated from human atherosclerotic intima. J Clin Invest 1997; 99:2075-2081.
14. Hazen S I, Crowley J R, Mueller D M, Heinecke J W. Mass spectrometric quantification of 3 chlorotyrosine in human tissue with attomole sensitivity: A sensitive and specific marker for myeloperoxidase-catalyzed chlorination at sites of inflammation. Free Rad Biol and Med 1997; 23:909-916.
15. Lavery J P & Miller C E. Deformation and creep in the human chorioamniotic sac. Am J Obstet Gynecol 1979; 134:366-375.
16. Masamoto N, Tasaka K, Mizuki J, Miyake A, Tanizawa O. Regulation of intracellular Mg2⁺ by superoxide in amnion cells. J Biochem and Biophys. Research Com 1992; 182:906-912.
17. Maymon E, Romero R, Pacora P, Gervasi M T, Gomez R, Edwin S S, Yoon B H. Evidence of in vivo differential bioavailability of the active forms of matrix metalloproteinases 9 and 2 in parturition, spontaneous rupture of membranes, and intra-amniotic infection. Am J Obstet Gynecol. 2000; 183 (4):887-94.
18. McGregor J A, French J I, Lawellin D, Franco-Buff A, Smith C, Todd J K. Bacterial protease-induced reduction of chorioamniotic membrane strength and elasticity. Obstet Gynecol 1987; 69:167-174.

19. McGregor J A, Schoonmaker J N, Lunt B D, Lawellin D W. Antibiotic inhibition of bacterially induced fetal membrane weakening. Obstet Gynecol. 1990; 76:124-128.
20. Michaelis J, Vissers M C, Winterbourn C C: Different effects of hypochlorous acid on human neutrophil metalloproteinases: Activation of collagenase andinactivation of collagenase and gelatinase. Arch Biochem Biophys 1992; 292:555-562.
21. Murray R K, Kelley F W. The extracellular matrix. In: Harper's Biochemistry. Chapter 57, p. 634-646, Appleton and Lange, Conn. 1993.
22. Plessinger M A, Woods J R, Miller R K. Pretreatment of human chorioamnion with vitamins C and E prevents hypochlorous acid-induced damage. Am J Obstet Gynecol. 2001; 185:5-10.
23. Pressman E, Woods J, Best. Tensile Strength of the Chorioamnion Throughout Gestation. Am J Obstet Gynecol. Abstract #294. 2001; 184: S92.
24. Rahman I and MacNee W. Role of oxidants/antioxidants in smoking-induced lung diseases. Free Radical Biology & Medicine. 1996; 21 (5):669-681.
25. Schoonmaker J N, Lawellin D W, Lunt B, McGregor J A. Bacteria and inflammatory cells reduce chorioamniotic membrane integrity and tensile strength. Obstet Gynecol 1989; 74:590-596.
26. So T. The role of matrix metalloproteinases for premature rupture of membranes. Nippon Sanka Fujinka Gakkai Zasshi-Acta. Obstet et Gynecol. Japonica 1993; 45:227-233.
27. Uchide K, Veno H. Inoue M, Sakai A, Fujimoto N & Okada Y. Matrix metalloproteinase-9 and tensile strength of fetal membranes in uncomplicated labor. Am J Obstet Gynecol 2000; 95:851-855.
28. Vadillo-Ortega F, Hernandez A, Gonzalez-Avila G, Bermejo L, Iwata K and Strauss J F (1996) Increased matrix metalloproteinase activity and reduced tissue inhibitor of metalloproteinase-1 level in amniotic fluids from pregnancies complicated by premature rupture of membranes. Am J Obstet Gynecol, I74, 1371-1376.
29. Wideman G L, Baird G H, Bolding O T: Ascorbic acid deficiency and premature rupture of fetal membranes. Am J Obstet Gynecol 1964; 88:592.
30. Winterbourn C C, Kettle A J. Biomarkers of myeloperoxidase-derived hypochlorous test. Free Rad Biol and Med 2000; 29:403-409.
31. Woessner J F. Matrix metalloproteinases and their inhibitors in connective tissue remodeling. FASEB J 1991; 5:2145-2154.
32. Woods J R. Reactive oxygen species and preterm premature rupture of membranes—A review. Placenta 2001; 22:Trophoblast Research 15:S38-S44.
33. Woods J R, Plessinger M A & Miller R K. Vitamins C and E: Missing links in preventing preterm premature rupture of membranes? Am J Obstet Gynecol. 2001; 185:5-10.
34. Woods J R, Cavanaugh J L, Norkus E P, Plessinger M A, Miller R K. Vitamins C and E in the human maternal-fetal unit at term. Abstract #158. Placenta 2001; 22:A48.
35. Woods J R, Cavanaugh J L, Plessinger M A, Norkus E, Miller R K. Labor depletes vitamin C in the mother and fetus but maintains vitamin E. Abstract #532. Am J Obstet Gynecol 2002; 185: S226.
36. Zavodnik I B, Lapshina E A, Zavodnik L B, Bartosz G, Soszynski M, Bryszewska M. Hypochlorous acid damages erythrocyte membrane proteins and alters lipid bilayer structure and fluidity. Free Rad Biol and Med 2001; 30:361-369.
37. Zimmerman E F, Potturi I B, Resnick E, Fisher J E. Role of oxygen free radicals in cocaine-induced vascular disruption in mice. Teratology 1994; 49:192-201.

We claim:

1. A method for determining the presence of hypochlorous acid in vaginal secretions comprising measuring the presence and amount of 3-chlorotyrosine in the vaginal secretions.

2. A method for determining the likelihood of preterm premature rupture of fetal membranes or preterm labor in a pregnant female comprising the steps of:
obtaining a sample of vaginal secretions from the female; and
analyzing the sample for the presence and amount of hypochlorous acid by measuring the amount of 3-chlorotyrosine in the sample.

3. A method for therapeutically treating a pregnant female to minimize the likelihood of preterm premature rupture of fetal membranes or preterm labor comprising the steps of:
obtaining a sample of vaginal secretions from the female;
measuring the presence and amount of 3-chlorotyrosine in the vaginal secretions wherein an increased amount of 3-chlorotyrosine represents an increased likelihood of preterm premature rupture of fetal membranes or preterm labor; and
administering an amount of dietary antioxidant to the female if the likelihood is increased.

4. The method of claim 3, wherein the dietary antioxidant is selected from the group consisting of vitamin C and vitamin E.

5. A method for determining the presence of hypochlorous acid in female vaginal secretions comprising measuring the presence and amount of 3-chlorotyrosine in the vaginal fluid using an ELISA assay.

6. A novel hapten for raising antibodies to 3-chlorotyrosine, said hapten comprising 3-(3-chloro-4-hydroxy-benzyl)-6-mercaptomethyl-piperazine-2,5-dione.

7. A neoantigen for raising antibodies to 3-chlorotyrosine comprising a carrier protein bound to the hapten of claim 6 by way of a covalent linkage.

8. The neoantigen of claim 7, wherein the carrier protein is selected from the group consisting of bovine serum albumin, keyhole limpet hemocyanin and thyroglobulin.

9. The neoantigen of claim 7, wherein the covalent linkage includes a sulfur atom.

10. A method for raising antibodies to 3-chlorotyrosine comprising the use of an antigen formed by covalently linking 3-(3-chloro-4-hydroxy-benzyl)-6-mercaptomethyl-piperazine-2,5-dione to a carrier protein.

11. The method of claim 10, wherein the carrier protein is selected from the group consisting of bovine serum albumin, keyhole limpet hemocyanin and thyroglobulin.

12. A method for raising antibodies to 3-chlorotyrosine comprising using an antigen formed by covalently linking N-acetyl-3-chlorotyrosine to a carrier protein.

13. The method of claim 12, further comprising using an antigen formed by covalently linking N-acetyl-3,5-dichlorotyprosine to a carrier protein.

14. The method of claim 12, wherein the carrier protein is selected from the group consisting of bovine serum albumin, keyhole limpet hemocyanin and thyroglobulin.
